(19) United States
(12) Patent Application Publication
(10) Pub. No.: US 2021/0249124 A1
(43) Pub. Date: Aug. 12, 2021

(54) Blockchain-Based Technologies for Medical Device Ecosystem

(71) Applicant: Mend Medical, LLC, Fort Wayne, IN (US)

(72) Inventors: Benjamin I Joseph, Grabill, IN (US); Bryce A. Isch, Warsaw, IN (US); Seth A. Nash, Fort Wayne, IN (US); Lindsey B. Tomer, Fort Wayne, IN (US)

(21) Appl. No.: 17/173,302

(22) Filed: Feb. 11, 2021

Related U.S. Application Data
(60) Provisional application No. 62/972,884, filed on Feb. 11, 2020.

(51) Int. Cl. G16H 40/40; G06Q 30/06

(52) U.S. Cl. CPC G16H 40/40; G06Q 30/0621

(57) ABSTRACT

Blockchain-based technologies for managing the manufacture and delivery of custom medical devices include a device availability manager, a medical device ordering manager and a distributed ledger module. The device availability manager is to generate medical device slot availability data for a plurality of device manufacturers. The medical device ordering manager is to order a custom medical device from one or more of the plurality of device manufacturers based on medical device slot availability data. The distributed ledger module is to create a block in a blockchain associated with a blockchain network that includes one or more of an identifier of the heathcare provider that ordered the custom medical device, and a prescription number.

### RELATED APPLICATIONS

This application claims the benefit of U.S. Provisional Application 62/972,884 filed Feb. 11, 2020 for “Blockchain-Based Technologies for Medical Device Ecosystem,” which is hereby incorporated by reference in its entirety.

### TECHNICAL FIELD

This disclosure relates generally to a blockchain-based ecosystem for leveraging manufacturing capabilities of multiple medical device manufacturers; in particular, this disclosure relates to an ecosystem that provides visibility, tracking, and traceability to the procurement and manufacture of custom medical devices.

### BACKGROUND

Within the medical device industry, there are circumstances in which custom and/or personalized medical devices may be necessary or desired. However, there can be difficulties in finding custom medical devices with the desired characteristics. Often, a healthcare provider must resort to calling potential device manufactures one-by-one to find a manufacturer for a desired custom device. One reason finding an appropriate custom device manufacturer can be difficult is regulatory limitations on how many custom devices a manufacturer can produce. In the U.S., the Food and Drug Administration (FDA) currently limits manufacturers to five (5) custom devices per year for a given device type. The regulatory audit process for these custom devices can also be time-consuming and burdensome on device manufacturers. These impediments to custom medical device procurement and tracking negatively impacts patient health because these important medical devices are not easily available to patients.

### SUMMARY

According to one aspect, this disclosure provides a computing device for managing the manufacture and delivery of custom medical devices using a blockchain network. The computing device includes a device availability manager to generate medical device slot availability data for a plurality of device manufacturers. The medical device slot availability data represents a number of custom medical devices that are available to be manufactured by the plurality of device manufacturers as a function of device type. The medical device ordering manager is to order a custom medical device from one or more of the plurality of device manufacturers based on medical device slot availability data and generate a prescription number that uniquely identifies the custom medical device to be manufactured. The computing device includes a distributed ledger module to create a block in a blockchain associated with a blockchain network. The block includes one or more of (i) an identifier of the heathcare provider that ordered the custom medical device, and (ii) the prescription number.

According to another aspect, this disclosure provides one or more non-transitory, computer-readable storage media comprising a plurality of instructions stored thereon that, in response to being executed, cause a computing device to: generate medical device slot availability data for a plurality of device manufacturers, wherein medical device slot availability data represents a number of custom medical devices that are available to be manufactured by the plurality of device manufacturers as a function of device type; order a custom medical device from one or more of the plurality of device manufacturers based on medical device slot availability data, wherein to order a custom medical device includes generating a prescription number that uniquely identifies the custom medical device to be manufactured; and create a block in a blockchain associated with a blockchain network, wherein the block includes one or more of (i) an identifier of the heathcare provider that ordered the custom medical device, and (ii) the prescription number.

According to a further aspect, this disclosure provides a method for managing the manufacture and delivery of custom medical devices using a blockchain network. The method includes the step of generating medical device slot availability data for a plurality of device manufacturers. The medical device slot availability data represents a number of custom medical devices that are available to be manufactured by the plurality of device manufacturers as a function of device type. A custom medical device is ordered from one or more of the plurality of device manufacturers based on medical device slot availability data; in response to the order, a prescription number is generated that uniquely identifies the custom medical device to be manufactured. The method includes creating a block in a blockchain associated with a blockchain network. The block includes one or more of (i) an identifier of the heathcare provider that ordered the custom medical device, and (ii) the prescription number.

### BRIEF DESCRIPTION OF THE DRAWINGS

The concepts described herein are illustrated by way of example and not by way of limitation in the accompanying figures. For simplicity and clarity of illustration, elements illustrated in the figures are not necessarily drawn to scale. Where considered appropriate, reference labels have been repeated among the figures to indicate corresponding or analogous elements.

FIG. 1 is a simplified block diagram of at least one embodiment of a blockchain-based system for medical device quality management and delivery;

FIG. 2 is a simplified block diagram of at least one embodiment of various environments of the system of FIG. 1;

FIGS. 3A-3D are tables illustrating a method of tracking available device slots according to at least one embodiment of the disclosure;

FIG. 4 is a simplified flow diagram of at least one embodiment of a method for determining available device slots;

FIGS. 5-6 are screenshots of an example user interface for the medical device search engine according to at least one embodiment of this disclosure;

FIG. 7 is a simplified flow diagram of at least one embodiment of a method for searching for an available custom medical device;

FIG. 8 is a simplified flow diagram of at least one embodiment of a method for processing an order for a custom medical device;

FIG. 9 is a simplified block diagram of at least one embodiment of a prescription number structure;

FIG. 10 is a simplified block diagram of at least one embodiment of a block in the blockchain created upon processing an order;

FIG. 11 is a simplified flow diagram of at least one embodiment of a method for monitoring development and manufacture of a custom medical device;

FIG. 12 is a simplified block diagram of at least one embodiment of a block in the blockchain created upon shipping an order for a custom medical device;

FIG. 13 is a simplified block diagram of at least one embodiment of an artifact for tracking the custom medical device;

FIG. 14 is a simplified flow diagram of at least one embodiment of a method for creating a medical device record for a custom medical device; and

FIG. 15 is a simplified block diagram of at least one embodiment of a block in the blockchain created upon creating a device medical record for a custom medical device.

### DETAILED DESCRIPTION OF THE DRAWINGS

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

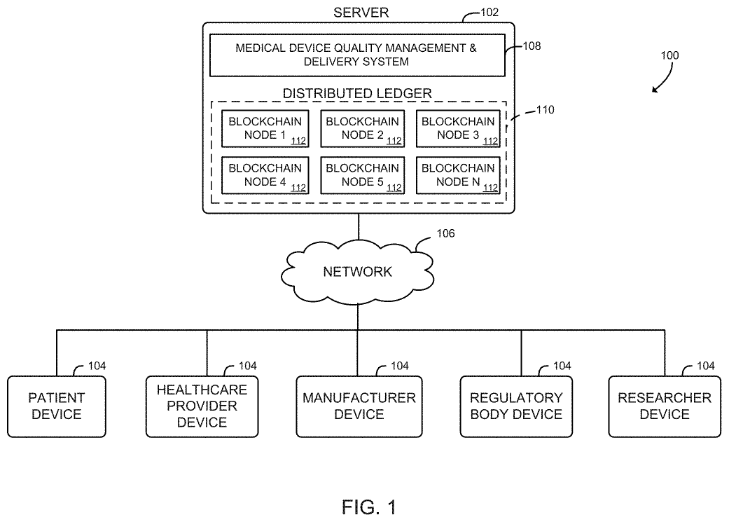

Referring now to FIG. 1, a blockchain-based system 100 for a medical device ecosystem includes one or more servers 102 in communication with one or more remote computing devices 104 over a network 106. Although a single server 102 is shown in FIG. 1 for purposes of example, one skilled in the art should appreciate that more than one server 102 could be used depending on the circumstances. For example, the server 102 could be a cloud-based platform with a plurality of servers accessible through the network 106.

The embodiment shown in FIG. 1 includes a plurality of remote computing devices 104 that are capable of accessing one or more functions of the server 102 over the network, but more or less remote computing devices 104 could be provided depending on the circumstances. In use, as described further herein, the system 100 provides transparency to allow real-time tracking of custom medical device records at the individual device/patient level. In some embodiments, the system 100 creates a secure, auditable independent record of device utilization and manufacturing traceability that could be accessed by regulators on demand, if needed. The system 100, in some embodiments, enables clinical trials and clinical studies in new ways by linking future data collection to a ledger of devices, clinicians, design, and manufacturing data. This can be done while providing for patient privacy and anonymity even though the device data is linked to individual patients. In some embodiments, the system captures any/all relevant communications relating to the creation of a custom medical device and permanently links these communications to the device record through blockchain technologies.

Although this system 100 is primarily described with regard to custom medical devices for purposes of example, it should be appreciated by one skilled in the art that certain aspects of the system 100 are applicable to non-custom medical devices (i.e., off-the-shelf medical devices).

In the embodiment shown in FIG. 1, the server 102 includes a medical device quality management and delivery system 108 and a distributed ledger 110 with a plurality of nodes 112. The medical device quality management and delivery system 108 includes features for, among other things, searching for custom medical devices, device management, tracking, and reporting capabilities as described herein. The distributed ledger 110 includes blockchain-based technologies to share and synchronize medical device record data across multiple nodes 112 that can be used to verify integrity of the data, such as for purposes of auditing. Typically, at least a portion of the distributed ledger 110 would be provided on a private blockchain network; however, embodiments are contemplated in which at least a portion of the distributed ledger 110 (or portions of data in the distributed ledger 110) could be publically available if the data were de-identified (e.g., cleaned of personal-identifiable data).

The server 102 and remote computing devices 104 may be embodied as any type of computation or computer device capable of performing the functions described herein, including, without limitation, a computer, a server, a workstation, a desktop computer, a laptop computer, a notebook computer, a tablet computer, a mobile computing device, a wearable computing device, a network appliance, a web appliance, a distributed computing system, a processor-based system, and/or a consumer electronic device. Additionally or alternatively, the server 102 may be embodied as a one or more compute sleds, memory sleds, or other racks, sleds, computing chassis, or other components of a physically disaggregated computing device. Depending on the circumstances, the server 102 and computing devices 104 could include a processor, an input/output subsystem, a memory, a data storage device, and/or other components and devices commonly found in a server or similar computing device. Of course, the server 102 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory, or portions thereof, may be incorporated in the processor in some embodiments.

The server 102 and computing devices 104 include a communication subsystem, which may be embodied as any communication circuit, device, or collection thereof, capable of enabling communications between the server 102 and remote computing devices 104 over the computer network 106. For example, the communication subsystem may be embodied as or otherwise include a network interface controller (NIC) or other network controller for sending and/or receiving network data with remote devices. The NIC may be embodied as any network interface card, network adapter, host fabric interface, network coprocessor, or other component that connects the server 102 and remote computing devices 104 to the network 106. The communication subsystem may be configured to use any one or more communication technology (e.g., wired or wireless communications) and associated protocols (e.g., Ethernet, InfiniBand®, Bluetooth®, Wi-Fi®, WiMAX, 3G, 4G LTE, etc.) to effect such communication.

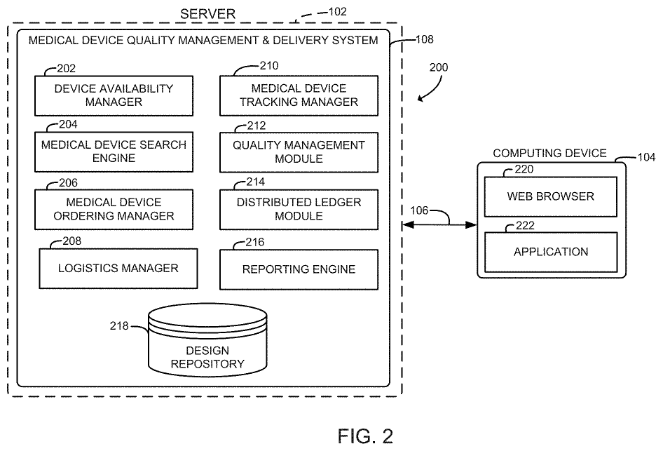

The remote computing devices 104 are configured to access one or more features of the server 102 over the network 106. For example, the server 102 may include a web-based interface or portal (see web browser 220 on FIG. 2) through which users of the remote computing devices 104 can interact with features of the server 102 using a browser, such as Chrome™ by Google, Inc. of Mountain View, Calif. Embodiments are also contemplated in which the remote computing devices 104 may be mobile devices running the Android™ operating system by Google, Inc. of Mountain View, Calif. and/or mobile devices running iOS™ operating system by Apple Inc. of Cupertino, Calif. on which software has been installed to perform one or more actions according to an embodiment of the present disclosure. For example, the computing devices 104 may have an app installed that allows a user to perform one or more actions described herein (see app 222 on FIG. 2). Although the system 100 is described as being a cloud-based platform accessible by the remote computing devices 104, in some embodiments one or more features of the server 102 could be performed locally on the remote computing devices 104.

Referring now to FIG. 2, in an illustrative embodiment, the server 102 establishes an environment 200 during operation for a medical device quality management and delivery system 108. The illustrative environment 200 includes a device availability manager 202, a medical device search engine 204, a medical device ordering manager 206, a logistics manager 208, a medical device tracking manager 210, a quality management module 212, a distributed ledger module 214, a reporting engine 216, and a design repository 218. As shown, the various components of the environment 200 may be embodied as hardware, firmware, software, or a combination thereof. As such, in some embodiments, one or more of the components of the environment 200 may be embodied as circuitry or collection of electrical devices (e.g., device availability manager circuitry 202, medical device search engine circuitry 204, a medical device ordering manager circuitry 206, a logistics manager circuitry 208, a medical device tracking manager circuitry 210, a quality management module circuitry 212, a distributed ledger module circuitry 214, a reporting engine circuitry 216). In the illustrative embodiment, the device availability manager circuitry 202, medical device search engine circuitry 204, medical device ordering manager circuitry 206, logistics manager circuitry 208, medical device tracking manager circuitry 210, quality management module circuitry 212, distributed ledger module circuitry 214, and reporting engine circuitry 216 are embodied as hardware, firmware, or other resources of the server 102. Additionally or alternatively, in some embodiments, those components may be embodied as hardware, firmware, or other resources of the computing device 104. Additionally, in some embodiments, one or more of the illustrative components may form a portion of another component and/or one or more of the illustrative components may be independent of one another.

The device availability manager 202 is configured to determine which medical device slots are available for each device type for each device manufacturer in the ecosystem. The device availability manager 202 is configured to determine available device slots for multiple manufacturers to leverage the available device slots for more than one device manufacturer. For custom medical devices, the terms “medical device slots” and “device slots” are intended to mean a number of custom medical devices for a particular device type that are available to be manufactured. The device availability manager 202 is configured to track the available medical device slots per device type per available device manufacturer in the ecosystem and aggregate these available medical device slots so the total medical device slots available can be tracked to provide visibility for ordering custom medical devices.

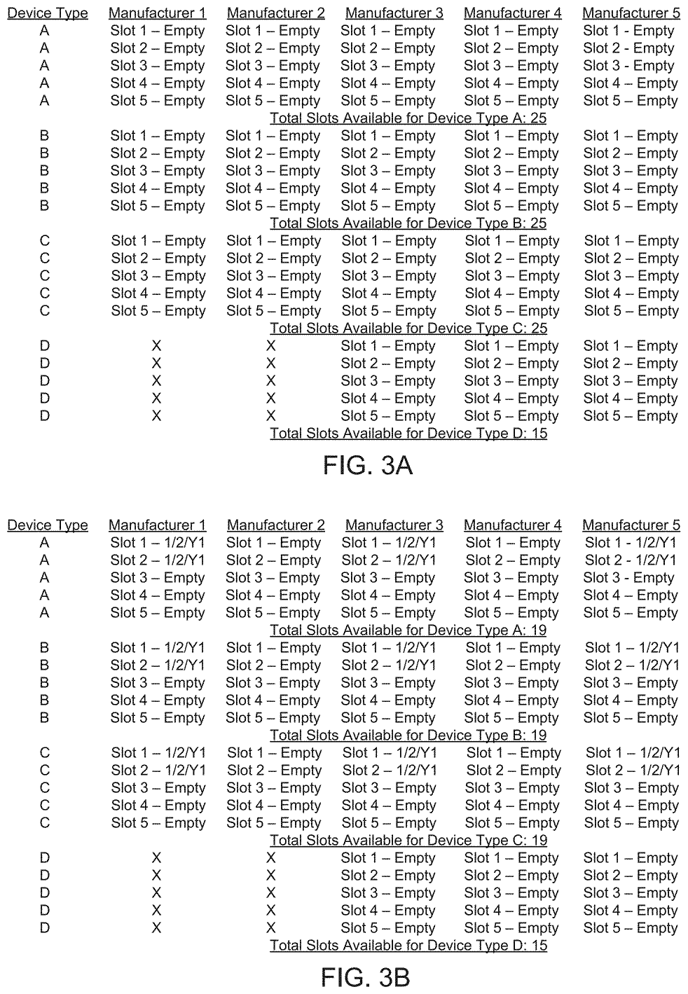

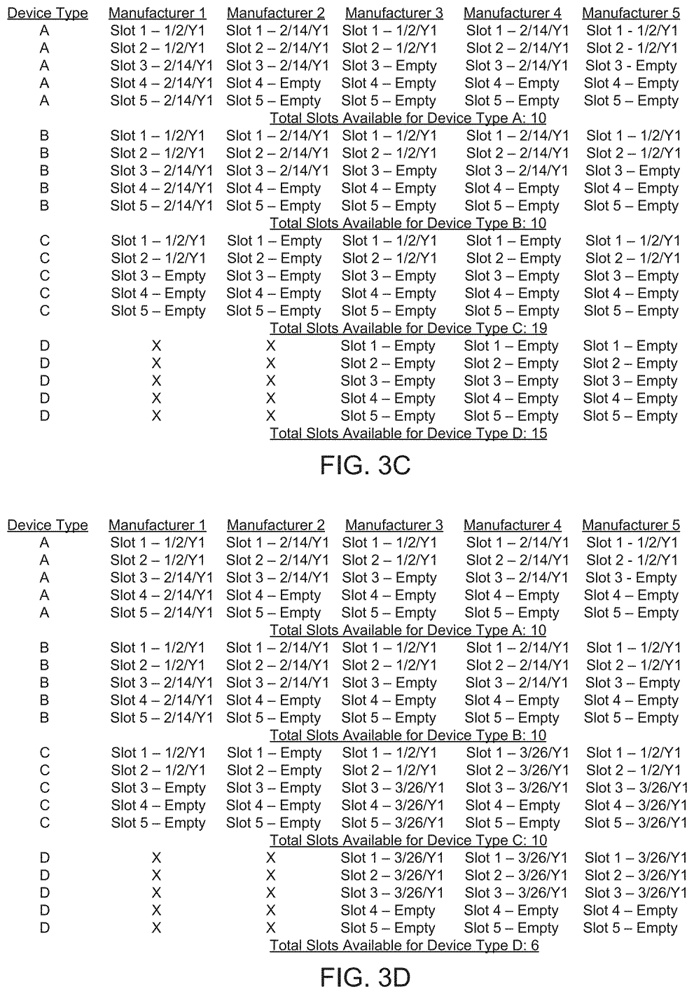

Consider an example in which the U.S. Food and Drug Administration (FDA) has a rule that prevents medical device manufacturers from manufacturing more than five (5) custom devices for a particular device category (as defined by the FDA) per year. Also, consider in this example that there are five (5) device manufacturers available in the ecosystem, with two of the device manufacturers (labeled as Manufacturers 1 and 2 in FIG. 3A) that manufacture in device types A, B, and C and the other three manufacturers (labeled as Manufacturers 3, 4 and 5 in FIG. 3A) that manufacture in device types A, B, C, and D. Initially, when all five manufacturers are added to the ecosystem, each could be set with five device slots available for each device category in which they manufacture, as illustrated in FIG. 3A. Of course, if any of the manufacturers have already used any device slots before entering the ecosystem, their available slots could be customized using the device availability manager 202.

Continuing with this example on FIG. 3B, consider on January 2 of Year 1 that Manufacturers 1, 3, and 5 obtain two (2) orders through the medical device ordering manager 206 (as described below) for device categories A, B, and C. When these orders are received through the medical device ordering manager 206, this is communicated to the device availability manager 202, which allocates device slots for these orders and tracks the available device slots both for individual manufacturers and in aggregate. FIG. 3B illustrates an embodiment of how the device availability manager 202 could track these orders to determine device slot availability.

FIG. 3C continues the example after, on February 14 of Year 1, Manufacturers 1, 2, and 4 receive three (3) orders for device categories A and B. The device availability manager 202 would allocate device slots for these orders and will track device slot availability as illustrated in FIG. 3C. Based on allocating device slots after the February 14 orders, the device availability manager 202 would be able to determine that Manufacturer 1 is out of device slots for device types A and B until January 2 of Year 2. Likewise, the device availability manager 202 would be able to determine that there are 10 available device slots in aggregate for all manufacturers in the ecosystem for device types A and B.

Continuing the example with regard to FIG. 3D, on March 26 of Year 1, Manufacturers 3, 4, and 5 receive three (3) orders for device categories C and D. The device availability manager 202 would allot three (3) device slots that are tagged with the date of March 26, Year 1 to slots for these respective manufacturers. As seen in FIG. 3D, the device availability manager 202 would be able to determine that Manufacturers 3 and 5 will no longer be able to accept orders for device type C after this order; accordingly, the device availability manager 202 would communicate with the medical device ordering module 206 to no longer accept orders for Manufacturers 3 and 5 for device type C until January 2 of Year 2. The device availability manager 202 would continue to manage device slots for each manufacturer in the ecosystem so there is visibility of total slots available for order, as well as ensuring that orders are not directed to manufacturers that are not currently able to manufacture the device type of that order.

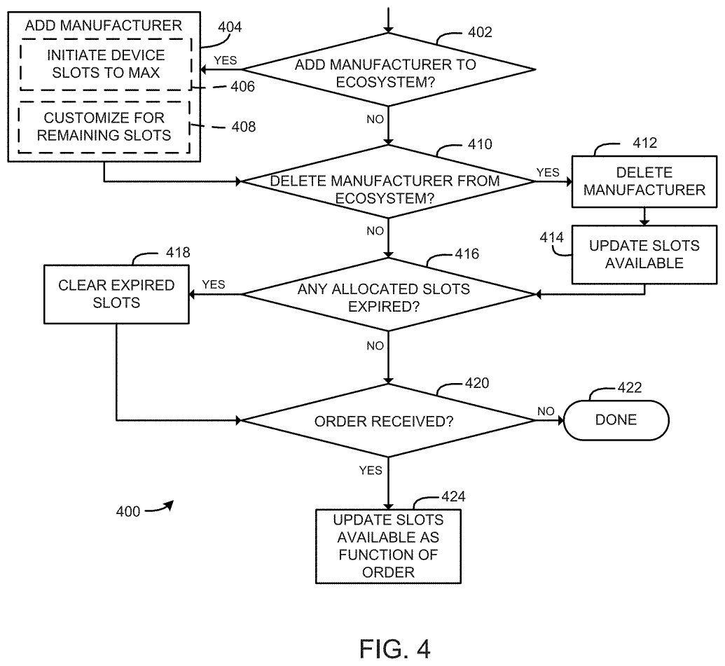

FIG. 4 illustrates a method 400 for managing device slot availability that may be executed by the server 102. It should be appreciated that, in some embodiments, the operations of the method 400 may be performed by one or more components of the environment 200 of the medical device quality management and delivery system 108 as shown in FIG. 2, such as the device availability manager 202. Also, it should be appreciated that the order of steps for the method 400 shown in FIG. 4 are for purposes of example, and could be in a different order; likewise, depending on the circumstances, some steps shown in the method 400 may be optional, or may not always be performed by the device availability manager 202. As illustrated, the method 400 begins in block 402 in which there is a determination whether any new manufacturers need to be added to the ecosystem. For example, the device availability manager 202 may include a user interface that allows management of the manufacturers that are included in the ecosystem; using this interface, new manufacturers could be added to the ecosystem and existing manufacturers could potentially be deleted. If no new manufacturers need to be added, the method advances to block 410. If one or more manufacturers need to be added to the ecosystem, the method 400 proceeds to block 404 in which the new manufacturer is added, which could include adding certain information about the new manufacturer in an ecosystem database. In some cases, if the new manufacturer has not already used device slots in the last year (or other regulatory period), the device slots for each device type are set to the regulatory maximum, which under current FDA rules would be five (block 406). If the new manufacturer has already used some of its device slots in the last year (or other regulatory period), the remaining device slots for the new manufacturer are customized through a user interface of the device availability manager 202 to reflect the remaining slots (block 408).

The method 400 then advances to block 410, in which there is a determination whether a manufacturer should be deleted from the ecosystem; if no manufacturer needs to be deleted (i.e., leave the ecosystem), the method 400 advances to block 416. If one or more manufacturers need to be deleted, the method 400 advances to block 412 in which a selected manufacturer is deleted from the ecosystem database, and in block 414, the remaining aggregate device slots for each device type is updated to delete the available slots attributed to the manufacturer that was deleted.

The method 400 next advances to block 416 in which there is a determination whether any currently allocated slots have expired. As discussed above, each manufacturer has a limited number of slots per device type per year (e.g., 5 slots/device type per year). Accordingly, as discussed above with regard to FIGS. 3A-3D, after the passage of sufficient time, device slots that had been allocate will again become available. The determination in block 416 determines whether any device slots are available again. If none of the allocated device slots have expired (i.e., none have become available again because sufficient time has passed), the method 400 advances to block 420. However, if some of the allocated device slots have expired, the method 400 advances to block 418 in which these expired device slots are cleared. In other words, the data regarding the expired device slots are updated to reflect that these device slots are available again.

Next, the method advances to block 420 in which a determination is made whether an order has been received; if not, the method 400 ends in block 422. If one or more orders have been received, however, the method 400 updates the available slots as a function of the device types in the orders (block 424). For example, each manufacturer for which an order is allocated has its device slots for that device type reduced as a function of the order. In other words, as explained with regard to FIGS. 3A-3D, a device slot is reduced for each device type in which a manufacturer receives an order and the order date is tracked so a determination can be made when the device slot is available again.

Referring again to FIG. 2, the medical device search engine 204 is configured to allow a user to search for available custom devices. In some embodiments, the medical device search engine 204 includes a user interface that allows a healthcare provider, such as a physician, to search for a desired custom medical device. For example, the user interface could include available device slots for multiple manufactures in the ecosystem based on device type.

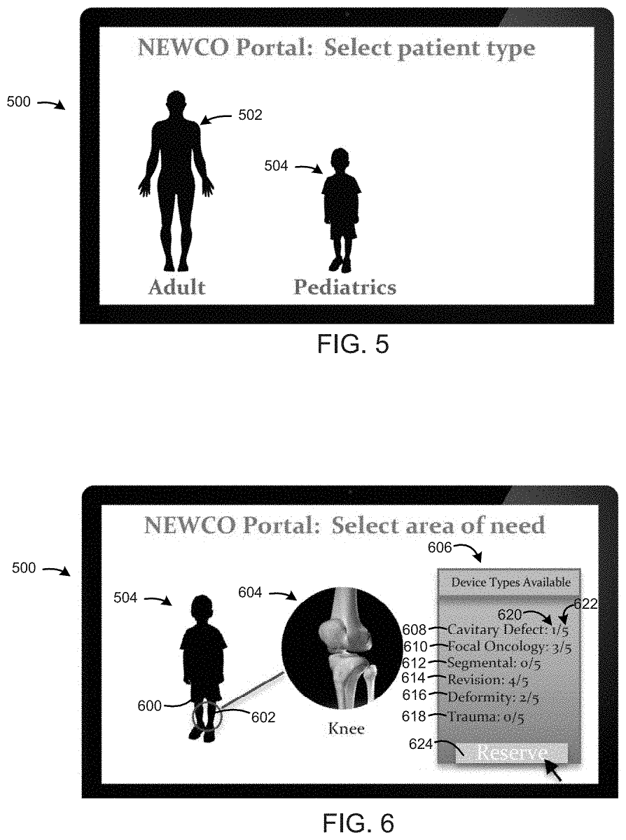

FIG. 5 shows an example user interface 500 from which a healthcare provider could search for available custom medical devices. This example user interface 500 would typically appear after a secure login by the user, such as by supplying a password, biometric data, security token, etc. The user can navigate the user interface 500 to order a custom medical device for a desired device type. As shown in FIG. 5, the user interface 500 allows the user to select between multiple different patient types, such as an adult patient 502 or a pediatric patient 504. Of course, these patient types are merely shown for purposes of example and other patient types (or other categories) could be provided on the user interface 500.

FIG. 6 shows the user interface 500 after the user has selected the pediatric patient category 504. As shown, the user is presented with a graphical representation 600 of the pediatric patient category 504. This graphical representation 600 allows the user to select an anatomical area of the patient for which a custom medical device is needed. In this example, the user has selected the knee area 602 of the graphical representation 600. As shown, the user interface 500 includes a more detailed graphical representation 604 of the selected area 602. Depending on the circumstances, this more detailed graphical representation 604 could provide further selection to further refine the anatomical area of need. As shown, the user interface 500 presents the available device types 606 for the selected knee area 602. In this example, the user interface 500 identifies the device types available as cavitary defect 608, focal oncology 610, segmental 612, revision 614, deformity 616, and trauma 618. The user is also presented with the number of device slots available 620 for each device type out of the total number of possible slots 622 for each device type. In this example, the user would be able to see there is 1 slot available for the cavitary defect device type 608, 3 slots available for the focal oncology device type 610, 4 slots available for the revision device type 612, and 2 slots available for the deformity device type. The user can also see that there is currently no available device slots for the segmental and trauma device types 612, 618.

Once the user selects the device type of interest, the user can select the “Reserve” button 624 to order a custom medical device. Depending on the circumstances, the user interface 500 could present the user with other information, including but not limited to price, description of device, and/or estimated delivery time. For device types in which there are currently no available device slots, the user interface 500 could include a date in which that device type will be available again based on when the next allocated device slot for that device type will expire. This information could be especially helpful for a healthcare provider if the allocated device slot for a device type of interest will expire in a short period of time, such as within the next couple weeks or month. Depending on the circumstances, the user interface 500 could include a “Contact Me When Available” button in which the healthcare provider could be sent a message when the allocated device slot expires and the device type of interest has a device slot that becomes available for manufacture.

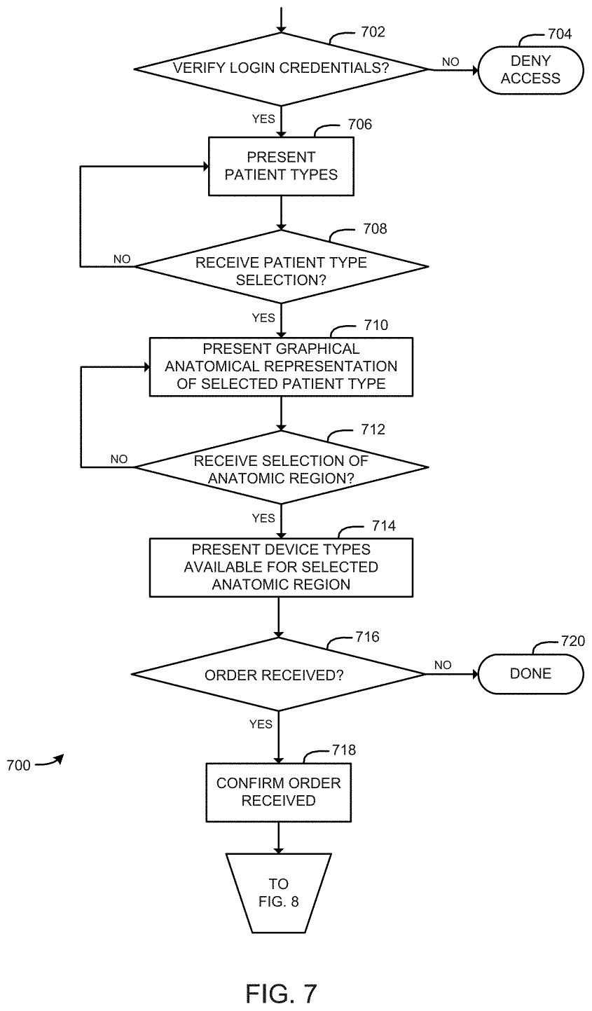

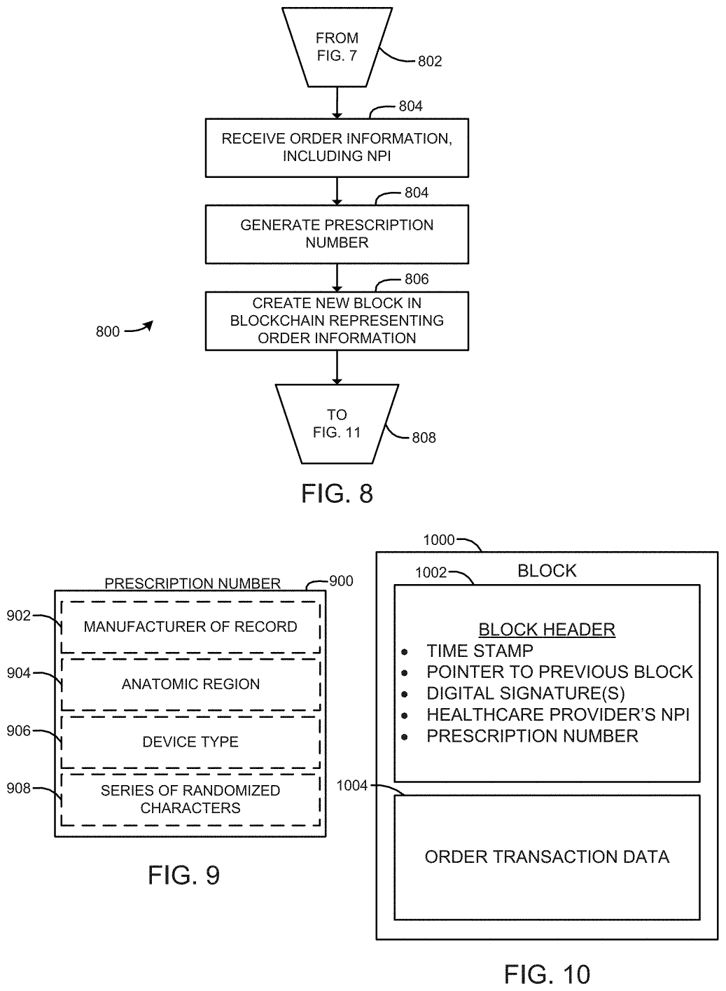

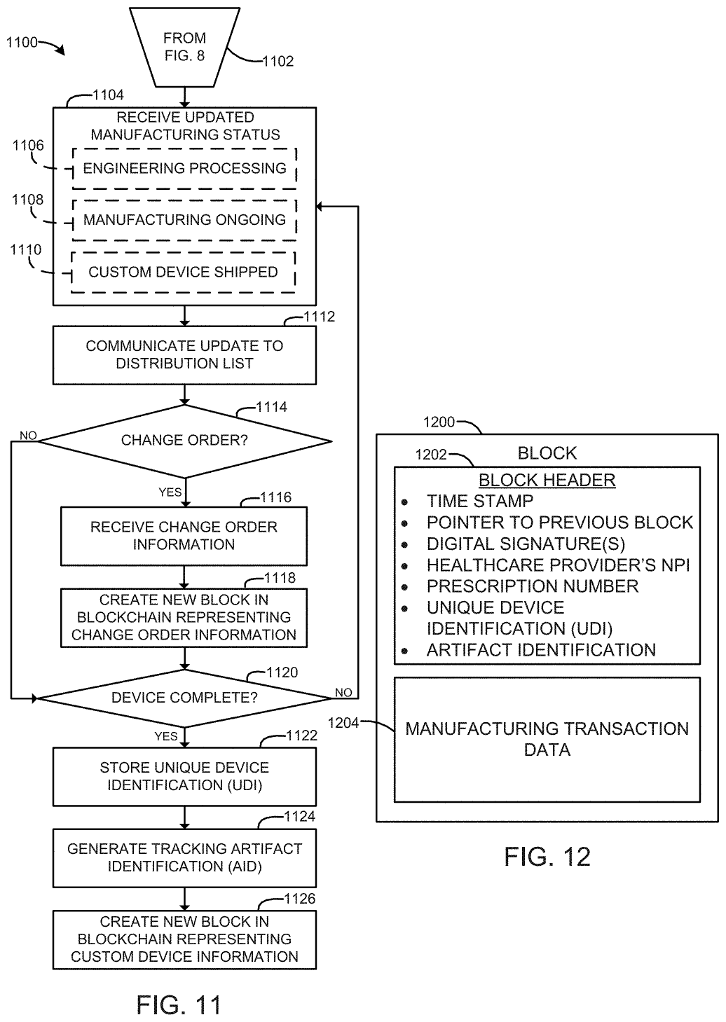

FIG. 7 illustrates a method 700 for searching availability of custom devices based on, among other possible things, device type and/or anatomic region that may be executed by the server 102. It should be appreciated that, in some embodiments, the operations of the method 700 may be performed by one or more components of the environment 200 of the medical device quality management and delivery system 108 as shown in FIG. 2, such as the medical device search engine 204. As shown, the method 700 starts at block 702 in which login credentials, including but not limited to a username/password, biometric data, and/or security token, are validated. If the login credentials cannot be validated, the method 700 advances to block 704 in which access to the search engine 204 is denied. If the login credentials are verified, the method 700 advances to block 706 in which a user interface, such as shown in FIG. 5, is presented to the user with a plurality of patient types. Upon receiving selection from the plurality of patient types (block 708), the method proceeds to block 710 in which the user interface 500 presents a graphical representation of the selected patient type. From the graphical representation, the user can select a particular anatomic region of interest. Upon receiving a selection of an anatomic region (block 712), the method 700 advances to block 714 in which the user is presented with a plurality of available device types for the selected anatomic region. As discussed above, the user may be presented with availability of the devices based on available device slots tracked by device availability manager 202, along with other information, such as price, device description, etc. The user can select a particular available device type to order through the user interface 500. If the user decides to order a custom medical device (block 716), the method 700 proceeds to block 718, in which the order is confirmed (block 718) and processed (see FIG. 8). Otherwise, if no order is made, the method 700 is complete (block 720).

The medical device ordering manager 206 is configured to, among other things, process orders received through the interface 500 and create an auditable trail using blockchain-based technology. FIG. 8 illustrates a method 800 that may, at least in part, be performed by the medical device ordering manager 206 on the server 102. As shown, the method 800 starts at block 802 in which an order has been received through the medical device search engine 204. The method advances to block 804 in which the device ordering manager 206 receives order information, including but not limited to the healthcare provider's national provider identifier (NPI). For example, the healthcare provider may have a profile on the system with NPI. Although the NPI would typically be used in the order information to identify the healthcare provider that ordered the custom medical device, the NPI is merely being used as an example and other information could be used to identify the healthcare provider in addition (or instead) of the NPI. By way of example only, depending on the circumstances, the healthcare provider could digitally sign the order for purposes of authenticating the order, in addition to the NPI (or as an alternative).

The method 800 then proceeds to block 804 in which a prescription number 900 (See FIG. 9) is generated. Although this example assumes the order will be fulfilled because the example user interface 500 only allows a healthcare provider to order if there is device slot available, other embodiments are contemplated in which availability to manufacture the custom device is determined after the healthcare provider requests the device; in such an embodiment, a prescription number may not be automatically generated upon receiving an order because the order might be denied if there is no device slot availability. Either way, the proscription number 900 is generated for the device upon verifying device slot availability.

Referring now to FIG. 9, the prescription number 900 is a new type of medical identifier that uniquely identifies a custom medical device in a structured manner. In the example shown, the prescription number 900 comprises both a hierarchy of information about the custom medical device and a series of randomized characters. As shown, the hierarchy of information includes a manufacturer of record 902 for the ordered medical device, an anatomic region 904 for the ordered medical device, and a device type 906 that identifies the ordered medical device by type (e.g., using the types identified by the FDA). Additionally, in the embodiment shown, the prescription number 900 includes a series of randomized characters 908. The term “randomized characters” is broadly intended to mean a unique data set, which could include alphanumeric characters, symbols, or other data. For example, an algorithm to generate a universally unique identifier (UUID) could be used to generate the series of randomized characters 908. Depending on the circumstances, in some embodiments, the randomized characters 908 could be a hash of a larger data set, such as the hash of the hierarchical information 902, 904, 906. By way of example only, if a manufacturer known as Indiana Orthopedic Company (IOC) is on this platform and the manufacturer of record 900, and the medical device ordering manager 206 receives an order from a healthcare provider for a custom device, and the requested device is for the shoulder (SHD) as the anatomic region 902, and is for a glenoid bone defect (GBD) as the device type 904, and the medical device ordering manager 206 generates a randomized 8 character number of X2$sox27 906, the assigned prescription number 908 for the order would be: IOC-SHD-GBD-X2$sox27.

The method 800 then proceeds to block 806 in which a new block is created in the blockchain. FIG. 10 illustrates an example block that could be added to the blockchain by the device ordering manager 206. In this example, the new block 1000 includes a block header 1002 and order transactional data 1004. As shown, the block header 1002 includes information that provides a chain of custody for the custom medical device. For example, the block header 1002 could include a time stamp when the block 1000 is created. If there are existing blocks in the blockchain (i.e., the new block is not the genesis block), the block header 1002 could include a pointer to the previous block in the blockchain. Depending on the circumstances, the block header 1002 could include a digital signature, such as the organization hosting the ecosystem for purposes of authenticating the block. The block header 1002 may also include the healthcare provider's NPI (or other identifying information of the healthcare provider), and the prescription number. This provides a technical advantage of transparently tracking the custom medical device based on the healthcare provider that ordered the device, along with the hierarchical information specific to the custom device in the prescription number. The block 1000 may also include a payload with transactional information about the order, such as price, description, etc. The method 800 then proceeds to block 808 in which the logistics manager 208 initiates communication with the device manufacturer to start development and manufacture of the custom device.

The medical device logistics manager 208 is configured to, among other things, provide manufacturing updates and create an auditable trail of the custom device's development and manufacturing process using blockchain-based technology. FIG. 11 illustrates a method 1100 that may, at least in part, be performed by the medical device logistics manager 208 on the server 102. As shown, the method 1100 starts at block 1102 in which order information has been received through the medical device ordering manager 206. The method 1100 proceeds to block 1104 in which updated information regarding the manufacturing process is received. There could be a plurality of predetermined stages from when the custom medical device is ordered to when the device ships to the healthcare provider. By way of example only, the predetermined stages could include that engineering to develop the device is processing 1106, manufacturing of the custom device is ongoing 1108, and/or that the custom device has been shipped 1110. For example, when one of these stages is reached, this could be entered into the medical device logistics manager 208, such as through a user interface. When one of these stages is reached, the method 1100 advances to block 1112 in which the new stage is communicated to a distribution list, such as the patient and/or healthcare provider. For example, the patient and/or healthcare provider could receive an email, text or other communication when the device is shipped and/or for other manufacturing and/or development updates. The method then proceeds to block 1114 in which there is a determination whether there is a change order. If there is no change order, the method 1100 proceeds to block 1120. If there is a change order, the method 1100 advances to block 1116 in which the change order information is received. Next, a new block is created in the blockchain with information regarding the change order information (block 1118). The method then proceeds to block 1120 in which there is a determination whether the device is complete. If the device is not complete, the method 1100 proceeds back to block 1104 to continue sending status updates on the development and manufacturing process. If the device is compete and ready for shipment, the method advances to block 1122 in which a unique device identification (UDI) for the custom device is stored in an ecosystem database.

The method 1100 then proceeds block 1124 in which an artifact identification (AID) for tracking the custom device is generated. As discussed below, the artifact is a computing device with one or more sensors, such as a GPS sensor, for tracking the geographical location of the custom device. The artifact could include an input/output interface, such as RFID or Bluetooth™ communications, from which the GPS position stored as the artifact travels between the manufacturer to the operating room can be read; in this way, the chain of custody between the manufacturer and operating room can be tracked. The AID is a unique identifier specific to the artifact that is associated with the custom device (or kit with other instruments used with the device).

The method then advances to block 1126 in which a new block is created in the blockchain with information about the custom device. FIG. 12 illustrates an example block that could be added to the blockchain by the medical device logistics manager 208. In this example, the new block 1200 includes a block header 1202 and manufacturing transactional data 1204. As shown, the block header 1202 includes information that provides a chain of custody for the custom medical device. For example, the block header 1202 could include a time stamp when the block 1200 is created. The block header 1202 could include a pointer to the previous block in the blockchain. Depending on the circumstances, the block header 1202 could include a digital signature, such as the organization providing the ecosystem for purposes of authenticating the block. The block header 1202 may also include the healthcare provider's NPI (or other identifying information of the healthcare provider), and the prescription number. The block header 1202 could also include the custom device's unique device identification (UDI) and the artifact identification (AID). This provides a technical advantage of transparently tracking the custom medical device based on the manufacturer that manufactured the device, along with information about the artifact that physically tracks the custom device. The block 1200 may also include a payload with transactional information about the manufacturer.

The medical device tracking manager 210 is configured to track the position of the custom device (and/or kit with other instruments for use with the custom device) to the healthcare provider and the operating room where the device is used. In some embodiments, the position of the custom device is tracked with an artifact that is placed in the package (or kit) in which the device is shipped. Embodiments are also contemplated in which the artifact could be attached, incorporated or integrated into the custom medical device itself.

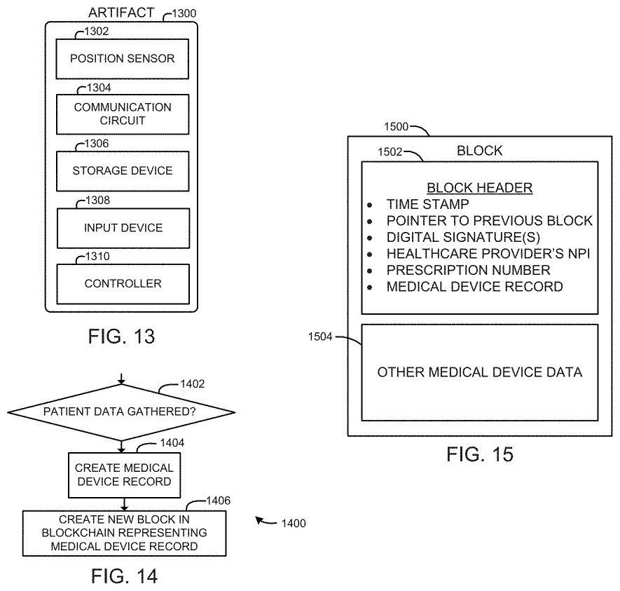

FIG. 13 illustrates an example artifact 1300 that goes with the package or kit that houses the custom medical device. For example, when a surgeon opens the package housing the kit that includes the custom device (along with other instruments used in the operating room to install the device), the artifact 1300 could be disposed within the package. In the embodiment shown, the artifact 1300 is a computing device with a position sensor 1302, a communication circuit 1304, a storage device 1306, an input device 1308, and a controller 1310. The storage device could be any electronic storage device configured to store data in a non-volatile manner. For example, the storage device 1306 could be flash memory and/or a solid-state drive (SSD). The controller 1310 could be any single or multicore processor and/or microcontroller that is configured to control operations of the artifact 1300.

The position sensor 1302 is configured to track the geographic position of the artifact. By having the artifact in the sealed package with the custom device (or embedded in the custom device itself), the position sensor 1302 facilitates position tracking of the custom device as the device travels from the manufacturer to the healthcare provider. In some embodiments, the position sensor 1302 could be a GPS sensor that tracks the geographic location of the artifact 1300. The location data of the position sensor 1302 is collected as the custom device travels between the manufacturer and operating room and stored in the storage device 1306.

The communication circuit 1304 is configured to receive and/or transmit information from the position sensor 1302 and/or data stored on the storage device 1306. For example, the storage device may have stored the artifact identification (AID) that uniquely identifies the artifact 1300; by way of example, the AID could be stored in a secure section of the storage device 1306 to prevent the AID from being altered or overwritten. In some embodiments, the communication circuit could include a RFID tag that is identified with the AID. Embodiments are contemplated in which the communication circuit could provide Bluetooth™, cellular, and/or other communications. For example, the artifact could periodically transmit position data and/or information entered through the input device 1308 to the server 102.

The input device 1308 could be a button, touch surface, keyboard, or other input device. The input device could be used by the user to input various information into the artifact 1300 or otherwise interact with the artifact 1300. For example, the input device 1308 could be used in the operating room to identify certain information about the use of the custom device. For example, in some embodiments, the input device 1308 could be a button that the surgeon presses in the operating room to establish a time in which the custom device was installed and that time would be stored in the storage device 1306. By way of another example, the input device 1308 could be a keyboard in which the user enters the number of unused instruments in the kit. After the custom device is installed (or post-surgery), in some embodiments, the artifact 1300 could be returned to the manufacturer or organizer of the ecosystem and the artifact 1300 scanned and the positional data (along with any other data entered through the input device 1308) can be stored in an ecosystem database and/or created as a block in the blockchain.

The quality management module 212 is configured to establish a medical device record for the custom device. The quality management module 212 is configured to, among other things, create a medical device record with certain data used in the design, development, and testing of the custom medical device. For example, the medical device record could include the data required by regulatory guidelines needed for an audit of the custom device. FIG. 14 illustrates a method 1400 that may, at least in part, be performed by the quality management module 212 on the server 102. The method 1400 starts at block 1402 in which the patient information needed to design and develop the custom device, such as patient imaging, is gathered. The method then advances to block 1404 in which the medical device record is created. The medical device record documents the custom device testing and validation, and could include design specifications, prints, CAD drawings, risk matrix, etc. These device testing and validation documents could be stored in the design depository 218. Upon completion design, testing and validation of the custom device, the method 1400 proceeds to block 1406 in which a block is created in the blockchain that represents the medical device record.

FIG. 15 illustrates an example block that could be created by the quality management module 212 to the blockchain. In this example, the new block 1500 includes a block header 1502 and manufacturing transactional data 1504. As shown, the block header 1502 includes information that provides a chain of custody for the custom medical device. For example, the block header 1502 could include a time stamp when the block 1500 is created. The block header 1502 could include a pointer to the previous block in the blockchain. Depending on the circumstances, the block header 1502 could include a digital signature, such as the organization hosting the ecosystem for purposes of authenticating the block. The block header 1502 may also include the healthcare provider's NPI (or other identifying information of the healthcare provider), and the prescription number. The block header could also include at least a portion of the medical device record or a hash of the medical device record. This provides a technical advantage of creating an unalterable medical device record that demonstrates testing and validation of the custom device. The block 1500 may also include a payload with other information about the testing and validation of the custom device.

Referring back to FIG. 2, the distributed ledger module 214 is configured to write blocks to the blockchain. In some embodiments, the distributed ledger module 214 could include a user interface for reading and/or searching blocks in the blockchain. In some cases, the distributed ledger module 214 may be configured to filter or de-identify patient data in the blockchain.

The reporting engine 216 is configured to provide access to read information in the blockchain (and/or other data in the ecosystem). For example, the system 100 could include a portal or user interface to access data from the blockchain. By way of example, the reporting engine 216 could be used by an auditor to audit, among other things, testing and validation of the custom medical device. For example, the reporting engine 216 could include a plurality of pre-formatted reports based on regulatory guidelines. In some cases, researchers could access the reporting engine 216 to aggregate data about custom devices in the ecosystem.

### EXAMPLES

Illustrative examples of the technologies disclosed herein are provided below. An embodiment of the technologies may include any one or more, and any combination of, the examples described below.

Example 1 is a computing device for managing the manufacture and delivery of custom medical devices using a blockchain network. The computing device includes a device availability manager to generate medical device slot availability data for a plurality of device manufacturers. The medical device slot availability data represents a number of custom medical devices that are available to be manufactured by the plurality of device manufacturers as a function of device type. The medical device ordering manager is to order a custom medical device from one or more of the plurality of device manufacturers based on medical device slot availability data and generate a prescription number that uniquely identifies the custom medical device to be manufactured. The computing device includes a distributed ledger module to create a block in a blockchain associated with a blockchain network. The block includes one or more of (i) an identifier of the heathcare provider that ordered the custom medical device, and (ii) the prescription number.

Example 2 includes the subject matter of Example 1, and wherein: to generate medical device slot availability data comprises to limit a number of medical device slots available to a predetermined device slot limit for each manufacturer as a function of device type for a predetermined period.

Example 3 includes the subject matter of Examples 1 and 2, and wherein: the medical device slot availability data comprises an aggregated total number of medical device slots available for the plurality of device manufacturers.

Example 4 includes the subject matter of Examples 1-3, and wherein: the device availability manager is to limit the number of custom medical devices that are available to be manufactured for each manufacturer of the plurality of device manufacturers to five medical devices per manufacturer per year per device type.

Example 5 includes the subject matter of Examples 1-4, and wherein: the prescription number comprises a plurality of randomized characters concatenated with a hierarchical structure that represents one or more of (i) a manufacturer of record, (ii) an anatomic region, or (iii) a device type.

Example 6 includes the subject matter of Examples 1-5, and further comprising a medical device search engine to search for a custom medical device as a function of one or more of (i) anatomic region or (ii) device type.

Example 7 includes the subject matter of Examples 1-6, and wherein: the medical device search engine is configured to generate search results representing one or more custom medical devices with medical device slot availability.

Example 8 includes the subject matter of Examples 1-7, and wherein: the search results include an aggregated total number of medical device slots available for the plurality of device manufacturers as a function of device type.

Example 9 includes the subject matter of Examples 1-8, and further comprising a logistics manager to generate an artifact identification that representing an identifier of a device that tracks a position of the custom medical device.

Example 10 includes the subject matter of Examples 1-9, and wherein: the distributed ledger module is to create a block in a blockchain associated with a blockchain network, wherein the block includes one or more of (i) an identifier of the heathcare provider that ordered the custom medical device, (ii) the prescription number, and (iii) the artifact identification.

Example 11 includes the subject matter of Examples 1-10, and further comprising a quality management module to create a medical device record for the custom medical device.

Example 12 includes the subject matter of Examples 1-11, and wherein: the distributed ledger module is to create a block in a blockchain associated with a blockchain network, wherein the block includes one or more of (i) an identifier of the heathcare provider that ordered the custom medical device, (ii) the prescription number, (iii) the artifact identification, and (iv) the medical device record.

Example 13 is one or more non-transitory, computer-readable storage media comprising a plurality of instructions stored thereon that, in response to being executed, cause a computing device to: generate medical device slot availability data for a plurality of device manufacturers, wherein medical device slot availability data represents a number of custom medical devices that are available to be manufactured by the plurality of device manufacturers as a function of device type; order a custom medical device from one or more of the plurality of device manufacturers based on medical device slot availability data, wherein to order a custom medical device includes generating a prescription number that uniquely identifies the custom medical device to be manufactured; and create a block in a blockchain associated with a blockchain network, wherein the block includes one or more of (i) an identifier of the heathcare provider that ordered the custom medical device, and (ii) the prescription number.

Example 14 includes the subject matter of Example 13, and wherein: to generate medical device slot availability data comprises to limit a number of medical device slots available to a predetermined device slot limit for each manufacturer as a function of device type for a predetermined period.

Example 15 includes the subject matter of Examples 13-14, and wherein: the medical device slot availability data comprises an aggregated total number of medical device slots available for the plurality of device manufacturers.

Example 16 includes the subject matter of Examples 13-15, and further comprising one or more instructions to limit the number of custom medical devices that are available to be manufactured for each manufacturer of the plurality of device manufacturers to five medical devices per manufacturer per year per device type.

Example 17 includes the subject matter of Examples 13-16, and wherein: the prescription number comprises a plurality of randomized characters concatenated with a hierarchical structure that represents one or more of (i) a manufacturer of record, (ii) an anatomic region, or (iii) a device type.

Example 18 includes the subject matter of Examples 13-17, and further comprising one or more instructions to search for a custom medical device as a function of one or more of (i) anatomic region or (ii) device type.

Example 19 includes the subject matter of Examples 13-18, and further comprising one or more instructions to generate search results representing one or more custom medical devices with medical device slot availability.

Example 20 includes the subject matter of Examples 13-19, and wherein: the search results include an aggregated total number of medical device slots available for the plurality of device manufacturers as a function of device type.

Example 21 includes the subject matter of Examples 13-20, and further comprising one or more instructions to generate an artifact identification that represents an identifier of a device that tracks a position of the custom medical device.

Example 22 includes the subject matter of Examples 13-21, and further comprising one or more instructions to create a block in a blockchain associated with a blockchain network, wherein the block includes one or more of (i) an identifier of the heathcare provider that ordered the custom medical device, (ii) the prescription number, and (iii) the artifact identification.

Example 23 includes the subject matter of Examples 13-22, and further comprising one or more instructions to create a medical device record for the custom medical device.

Example 24 includes the subject matter of Examples 13-23, and further comprising one or more instructions to create a block in a blockchain associated with a blockchain network, wherein the block includes one or more of (i) an identifier of the heathcare provider that ordered the custom medical device, (ii) the prescription number, (iii) the artifact identification, and (iv) the medical device record.

Example 25 is a method for managing the manufacture and delivery of custom medical devices using a blockchain network. The method includes the step of generating medical device slot availability data for a plurality of device manufacturers. The medical device slot availability data represents a number of custom medical devices that are available to be manufactured by the plurality of device manufacturers as a function of device type. A custom medical device is ordered from one or more of the plurality of device manufacturers based on medical device slot availability data; in response to the order, the method includes generating a prescription number that uniquely identifies the custom medical device to be manufactured. The method includes creating a block in a blockchain associated with a blockchain network. The block includes one or more of (i) an identifier of the heathcare provider that ordered the custom medical device, and (ii) the prescription number.

Example 26 includes the subject matter of Example 25, and wherein: generating medical device slot availability data comprises to limit a number of medical device slots available to a predetermined device slot limit for each manufacturer as a function of device type for a predetermined period.

Example 27 includes the subject matter of Examples 25-26, and wherein: the medical device slot availability data comprises an aggregated total number of medical device slots available for the plurality of device manufacturers.

Example 28 includes the subject matter of Examples 25-27, and further comprising limiting the number of custom medical devices that are available to be manufactured for each manufacturer of the plurality of device manufacturers to five medical devices per manufacturer per year per device type.

Example 29 includes the subject matter of Examples 25-28, and wherein: the prescription number comprises a plurality of randomized characters concatenated with a hierarchical structure that represents one or more of (i) a manufacturer of record, (ii) an anatomic region, or (iii) a device type.

Example 30 includes the subject matter of Examples 25-29, and further comprising searching for a custom medical device as a function of one or more of (i) anatomic region or (ii) device type.

Example 31 includes the subject matter of Examples 25-30, and further comprising generating search results representing one or more custom medical devices with medical device slot availability.

Example 32 includes the subject matter of Examples 25-31, and wherein: the search results include an aggregated total number of medical device slots available for the plurality of device manufacturers as a function of device type.

Example 33 includes the subject matter of Examples 25-32, and further comprising generating an artifact identification that representing an identifier of a device that tracks a position of the custom medical device.

Example 34 includes the subject matter of Examples 25-33, and further comprising creating a block in a blockchain associated with a blockchain network, wherein the block includes one or more of (i) an identifier of the heathcare provider that ordered the custom medical device, (ii) the prescription number, and (iii) the artifact identification.

Example 35 includes the subject matter of Examples 25-34, and further comprising creating a medical device record for the custom medical device.

Example 36 includes the subject matter of Examples 25-35, and further comprising creating a block in a blockchain associated with a blockchain network, wherein the block includes one or more of (i) an identifier of the heathcare provider that ordered the custom medical device, (ii) the prescription number, (iii) the artifact identification, and (iv) the medical device record.

Example 37 is a system for managing the manufacture and delivery of custom medical devices using a blockchain network. The system includes means for generating medical device slot availability data for a plurality of device manufacturers. The medical device slot availability data represents a number of custom medical devices that are available to be manufactured by the plurality of device manufacturers as a function of device type. The system includes means for ordering a custom medical device from one or more of the plurality of device manufacturers based on medical device slot availability data; in response to the order, the system includes means for generating a prescription number that uniquely identifies the custom medical device to be manufactured. The system includes means for creating a block in a blockchain associated with a blockchain network. The block includes one or more of (i) an identifier of the heathcare provider that ordered the custom medical device, and (ii) the prescription number.

1. A computing device for managing the manufacture and delivery of custom medical devices using a blockchain network, the computing device comprising:
a device availability manager to generate medical device slot availability data for a plurality of device manufacturers, wherein medical device slot availability data represents a number of custom medical devices that are available to be manufactured by the plurality of device manufacturers as a function of device type;
a medical device ordering manager to order a custom medical device from one or more of the plurality of device manufacturers based on medical device slot availability data, wherein to order a custom medical device includes generating a prescription number that uniquely identifies the custom medical device to be manufactured; and
a distributed ledger module to create a block in a blockchain associated with a blockchain network, wherein the block includes one or more of (i) an identifier of the heathcare provider that ordered the custom medical device, and (ii) the prescription number.

2. The computing device of claim 1, wherein to generate medical device slot availability data comprises to limit a number of medical device slots available to a predetermined device slot limit for each manufacturer as a function of device type for a predetermined period.

3. The computing device of claim 1, wherein the medical device slot availability data comprises an aggregated total number of medical device slots available for the plurality of device manufacturers.

4. The computing device of claim 1, wherein the device availability manager is to limit the number of custom medical devices that are available to be manufactured for each manufacturer of the plurality of device manufacturers to five medical devices per manufacturer per year per device type.

5. The computing device of claim 1, wherein the prescription number comprises a plurality of randomized characters concatenated with a hierarchical structure that represents one or more of (i) a manufacturer of record, (ii) an anatomic region, or (iii) a device type.

6. The computing device of claim 1, further comprising a medical device search engine to search for a custom medical device as a function of one or more of (i) anatomic region or (ii) device type.

7. The computing device of claim 6, wherein the medical device search engine is configured to generate search results representing one or more custom medical devices with medical device slot availability.

8. The computing device of claim 6, wherein the search results include an aggregated total number of medical device slots available for the plurality of device manufacturers as a function of device type.

9. The computing device of claim 1, further comprising a logistics manager to generate an artifact identification that representing an identifier of a device that tracks a position of the custom medical device.

10. The computing device of claim 9, wherein the distributed ledger module is to create a block in a blockchain associated with a blockchain network, wherein the block includes one or more of (i) an identifier of the heathcare provider that ordered the custom medical device, (ii) the prescription number, and (iii) the artifact identification.

11. The computing device of claim 1, further comprising a quality management module to create a medical device record for the custom medical device.

12. The computing device of claim 11, wherein the distributed ledger module is to create a block in a blockchain associated with a blockchain network, wherein the block includes one or more of (i) an identifier of the heathcare provider that ordered the custom medical device, (ii) the prescription number, (iii) the artifact identification, and (iv) the medical device record.

13. One or more non-transitory, computer-readable storage media comprising a plurality of instructions stored thereon that, in response to being executed, cause a computing device to:
generate medical device slot availability data for a plurality of device manufacturers, wherein medical device slot availability data represents a number of custom medical devices that are available to be manufactured by the plurality of device manufacturers as a function of device type;
order a custom medical device from one or more of the plurality of device manufacturers based on medical device slot availability data, wherein to order a custom medical device includes generating a prescription number that uniquely identifies the custom medical device to be manufactured; and
create a block in a blockchain associated with a blockchain network, wherein the block includes one or more of (i) an identifier of the heathcare provider that ordered the custom medical device, and (ii) the prescription number.

14. The one or more non-transitory, computer-readable storage media of claim 13, wherein to generate medical device slot availability data comprises to limit a number of medical device slots available to a predetermined device slot limit for each manufacturer as a function of device type for a predetermined period.

15. The one or more non-transitory, computer-readable storage media of claim 13, wherein the medical device slot availability data comprises an aggregated total number of medical device slots available for the plurality of device manufacturers.

16. The one or more non-transitory, computer-readable storage media of claim 13, further comprising one or more instructions to limit the number of custom medical devices that are available to be manufactured for each manufacturer of the plurality of device manufacturers to five medical devices per manufacturer per year per device type.

17. The one or more non-transitory, computer-readable storage media of claim 13, wherein the prescription number comprises a plurality of randomized characters concatenated with a hierarchical structure that represents one or more of (i) a manufacturer of record, (ii) an anatomic region, or (iii) a device type.

18. The one or more non-transitory, computer-readable storage media of claim 13, further comprising one or more instructions to search for a custom medical device as a function of one or more of (i) anatomic region or (ii) device type.

19. The one or more non-transitory, computer-readable storage media of claim 18, further comprising one or more instructions to generate search results representing one or more custom medical devices with medical device slot availability.

20. The one or more non-transitory, computer-readable storage media of claim 18, wherein the search results include an aggregated total number of medical device slots available for the plurality of device manufacturers as a function of device type.

21. The one or more non-transitory, computer-readable storage media of claim 13, further comprising one or more instructions to generate an artifact identification that represents an identifier of a device that tracks a position of the custom medical device.

22. The one or more non-transitory, computer-readable storage media of claim 21, further comprising one or more instructions to create a block in a blockchain associated with a blockchain network, wherein the block includes one or more of (i) an identifier of the heathcare provider that ordered the custom medical device, (ii) the prescription number, and (iii) the artifact identification.

23. The one or more non-transitory, computer-readable storage media of claim 13, further comprising one or more instructions to create a medical device record for the custom medical device.

24. The one or more non-transitory, computer-readable storage media of claim 23, further comprising one or more instructions to create a block in a blockchain associated with a blockchain network, wherein the block includes one or more of (i) an identifier of the heathcare provider that ordered the custom medical device, (ii) the prescription number, (iii) the artifact identification, and (iv) the medical device record.

25. A method for managing the manufacture and delivery of custom medical devices using a blockchain network, the method comprising:
generating medical device slot availability data for a plurality of device manufacturers, wherein medical device slot availability data represents a number of custom medical devices that are available to be manufactured by the plurality of device manufacturers as a function of device type;
ordering a custom medical device from one or more of the plurality of device manufacturers based on medical device slot availability data, wherein to order a custom medical device includes generating a prescription number that uniquely identifies the custom medical device to be manufactured; and
creating a block in a blockchain associated with a blockchain network, wherein the block includes one or more of (i) an identifier of the heathcare provider that ordered the custom medical device, and (ii) the prescription number.

26. The method of claim 25, wherein generating medical device slot availability data comprises to limit a number of medical device slots available to a predetermined device slot limit for each manufacturer as a function of device type for a predetermined period.

27. The method of claim 25, wherein the medical device slot availability data comprises an aggregated total number of medical device slots available for the plurality of device manufacturers.

28. The method of claim 25, wherein further comprising limiting the number of custom medical devices that are available to be manufactured for each manufacturer of the plurality of device manufacturers to five medical devices per manufacturer per year per device type.

29. The method of claim 25, wherein the prescription number comprises a plurality of randomized characters concatenated with a hierarchical structure that represents one or more of (i) a manufacturer of record, (ii) an anatomic region, or (iii) a device type.

30. The method of claim 25, further comprising searching for a custom medical device as a function of one or more of (i) anatomic region or (ii) device type.

31. The method of claim 30, further comprising generating search results representing one or more custom medical devices with medical device slot availability.

32. The method of claim 30, wherein the search results include an aggregated total number of medical device slots available for the plurality of device manufacturers as a function of device type.

33. The method of claim 25, further comprising generating an artifact identification that representing an identifier of a device that tracks a position of the custom medical device.

34. The method of claim 33, further comprising creating a block in a blockchain associated with a blockchain network, wherein the block includes one or more of (i) an identifier of the heathcare provider that ordered the custom medical device, (ii) the prescription number, and (iii) the artifact identification.

35. The method of claim 25, further comprising creating a medical device record for the custom medical device.

36. The method of claim 35, further comprising creating a block in a blockchain associated with a blockchain network, wherein the block includes one or more of (i) an identifier of the heathcare provider that ordered the custom medical device, (ii) the prescription number, (iii) the artifact identification, and (iv) the medical device record.
